(19) United States
(12) Patent Application Publication
(10) Pub. No.: US 2024/0407760 A1
(43) Pub. Date: Dec. 12, 2024

(54) METHOD AND APPARATUS FOR MEASURING NEUROVASCULAR COUPLING

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); ECOLE SUPÉRIEURE DE PHYSIQUE ET DE CHIMIE INDUSTRIELLES DE LA VILLE DE PARIS, Paris (FR)

(72) Inventors: Mickael TANTER, Paris (FR); Thomas DEFFIEUX, Paris (FR); Franck LEBRIN, Paris (FR); Clémentine MORISSET, Paris (FR)

(21) Appl. No.: 18/698,480

(22) PCT Filed: Oct. 12, 2022
(86) PCT No.: PCT/EP2022/078398

§ 371 Date: Apr. 4, 2024

(30) Foreign Application Priority Data
Oct. 12, 2021 (EP) 21306428.0

(51) Int. Cl. A61B 8/08; A61B 8/10

(52) U.S. Cl. CPC A61B 8/488; A61B 8/10; A61B 8/5292

(57) ABSTRACT

Functional imaging, in particular functional ultrasound imaging, is becoming a powerful tool for early detection of disorders such as neurodegenerative diseases. The present disclosure proposes a reliable method for such early detection, by delivering a stimulus to the nervous system, performing a functional imaging of an area of interest of the nervous system activated by the stimulus to obtain a series of hemodynamic Doppler images of the vascular network in the area of interest, and computing, from the series of hemodynamic Doppler images, a hemodynamic response (22) to the stimulus. The shape of hemodynamic response may be used to detect health disorders.

### TECHNICAL FIELD

The present disclosure concerns methods and apparatuses for measuring neurovascular coupling in a nervous system of a human or animal.

### BACKGROUND ART

Functional imaging enables to assess nervous activity in the nervous system based on the activity of the vascular network in said nervous system. This is based on the phenomenon of neurovascular coupling: activated zones of the nervous system need more oxygen, thus locally increasing the flow of blood in the vascular network of said nervous system, in particular in the capillaries, venules and arterioles of the vascular network.

One type of functional imaging of high interest, particularly regarding efficiency and cost, is ultrasound functional imaging, especially based on ultrafast ultrasound imaging. Such technique has been described in particular by Macé et al. [E. Mace, G. Montaldo, B. Osmanski, I. Cohen, M. Fink and M. Tanter, “Functional ultrasound imaging of the brain: theory and basic principles,” in IEEE Transactions on Ultrasonics, Ferroelectrics, and Frequency Control, vol. 60, no. 3, pp. 492-506, March 2013].

Recent neuroimaging studies show that assessing the neurovascular coupling is a potential way for early screening and monitoring of disorders, particularly cardiovascular or neurodegenerative disorders, including:
Alzheimer's disease [Iadecola, C. Neurovascular regulation in the normal brain and in Alzheimer's disease. Nat Rev Neurosci 5, 347-360 (2004)] [Kisler, K., Nelson, A. R., Montagne, A. & Zlokovic, B. V. Cerebral blood flow regulation and neurovascular dysfunction in Alzheimer disease. Nat Rev Neurosci 18, 419-434 (2017)] [Zlokovic, Neurovascular mechanisms of Alzheimer's neurodegeneration, Trends Neurosci (2005)], [Kisler, K., Nelson, A. R., Montagne, A., and Zlokovic, B. V. Cerebral blood flow regulation and neurovascular dysfunction in Alzheimer disease. Nat. Rev. Neurosci. 18, 419-434 (2017)],
hypertension [Girouard, H. & Iadecola, C. Neurovascular coupling in the normal brain and in hypertension, stroke, and Alzheimer disease. Journal of Applied Physiology 100, 328-335 (2006)],
ischemic stroke [del Zoppo, The neurovascular unit in the setting of stroke, J Intern Med 267:156-171 (2010)],
amyotrophic lateral sclerosis [Murphy, M. J., Grace, G. M., Tartaglia, M. C., Orange, J. B., Chen, X., Rowe, A., Findlater, K., Kozak, R. I., Freedman, M., Lee, T.-Y., and Strong, M. J. (2012) Widespread cerebral hemodynamics disturbances occur early in amyotrophic lateral sclerosis. Amyotroph. Lateral Scler. 13, 202-209.], and
obesity [Tucsek Z, Toth P, Tarantini S, Sosnowska D, Gautam T, Warrington J P, Giles C B, Wren J D, Koller A, Ballabh P, Sonntag W E, Ungvari Z, Csiszar A, (2014), Aging exacerbates obesity-induced cerebromicrovascular rarefaction, neurovascular uncoupling, and cognitive decline in mice. J Gerontol A Biol Sci Med Sci 69:1339-1352.].

One purpose of the present disclosure is to provide a method for measuring neurovascular coupling in a nervous system of a human or animal, which would provide precise and reliable biomarkers for health disorders, in particular some neurogenerative and cardiovascular diseases.

### SUMMARY

To this end, the present disclosure proposes a method for measuring neurovascular coupling in a nervous system of a human or animal, said nervous system having a vascular network, said method including:
(a) delivering at least one stimulus to said nervous system, said stimulus activating said nervous system in at least one region thereof, which in turn causes a hemodynamic response in said vascular network in said region;
(b) performing a series of at least 10 ultrasound measurements of said region with an ultrasound probe having an array of at least one ultrasound transducer, to obtain hemodynamic Doppler samples of said vascular network in said region, during a recording period of at least 10 seconds including said stimulus, each Doppler sample having a certain Doppler signal;
(c) computing, from said series of hemodynamic Doppler measurements, a hemodynamic response to said stimulus in at least one area of interest in said region during said recording period, said hemodynamic response including values of at least one hemodynamic parameter in said vascular network based on said Doppler signals of said Doppler samples of said series.

The present inventors determined that the shape of the hemodynamic response can be used as a reliable biomarker of certain health disorders. Thus, based on the hemodynamic response, it is possible to reliably determine whether the neurovascular coupling is normal or not, and to use the neurovascular coupling as a biomarker of diseases such as certain neurodegenerative or cardiovascular diseases.

In embodiments of the method, one may use the following features, alone or in combination:
said area of interest is determined automatically based on an activation map of said vascular network, estimated from correlation of said Doppler signals with said stimulus;
said area of interest is determined automatically based on Doppler intensity of the Doppler signals;
said area of interest is determined automatically based on a B-mode image if said nervous system is a retina;
said area of interest is determined automatically based on an external neuronavigation device;
said ultrasound measurements are Doppler images and said Doppler samples are pixels of said Doppler images;
said ultrasound measurements correspond to one or several lines in a direction of depth from said ultrasound probe;
an interrogation ultrasonic beam transmitted by the ultrasound probe is moved between measurements to scan at least part of said region;
said series of ultrasound measurements is performed at a rate of at least 1 Doppler image per second;
during said recording period, said series of hemodynamic ultrasound measurements includes at least 50 ultrasound measurements, in particular at least 100 ultrasound measurements;
said recording period is of at least 5 seconds, for instance at least 7 seconds after said stimulus;
said recording period is of at least 5 seconds before and after said stimulus, for instance of at least 7 seconds before and after said stimulus;
said recording period is of at most 20 seconds before and after said stimulus, for instance of at most 10 seconds before and after said stimulus;
said hemodynamic parameter is the Doppler signal;
said hemodynamic parameter is a relative variation of the Doppler signal in said area of interest, relative to a baseline of said Doppler signal;
said area of interest is determined based on a preexisting functional map of said region;
said area of interest includes at least a pixel of maximum correlation with the stimulus;
said area of interest is constituted by said pixel of maximum correlation and a predetermined number of additional pixels around said pixel of maximum correlation;
said predetermined number of additional pixels is comprised within a radius of 1 to 6 pixels around said pixel of maximum response, for instance within a radius of 2 to 4 pixels;
said hemodynamic response is averaged on said area of interest;
said stimulus has a stimulus duration comprised between 0.05 and 60 seconds, in particular between 0.5 and 1 second, for instance 0.8 seconds;
said stimulus is sensorial, in particular one of: an optical stimulus transmitted through at least one eye, an auditive stimulus transmitted through at least one ear, an odor stimulus transmitted through the nose, a taste stimulus transmitted through the mouth, a contact or shock or electrical stimulus transmitted in particular through the skin;
said steps (a) and (b) are repeated for n trials and said hemodynamic response is averaged on said n trials, n being an integer larger than 1;
said steps (a) and (b) are repeated for n trials and said hemodynamic response is used to assess a reproducibility parameter or a quality parameter on said n trials, n being an integer larger than 1;
n is comprised between 10 and 100, for instance comprised between 20 and 60, in particular comprised between 20 and 30;
said array is one of a single transducer, a few transducers (e.g. less than 10), a linear array of transducers (1D matrix), a 2D matrix of transducers and a sparce matrix of transducers;
said ultrasound measurements are based on ultrasensitive Doppler or ultrafast ultrasound imaging with a Pulse Repetition Frequency over 500 Hz;
said ultrasound measurements are based on unfocused ultrasound waves;
said ultrasound measurements are controlled by signals from at least one external device such as video, EEG, ECG, detector of movement of the animal or patient;
said Doppler samples are obtained by one of: standard Doppler and micro-Doppler (see above article of Macé, 2013);
said Doppler signal is based on one of power Doppler, color Doppler, index of vascular resistivity, or any combination thereof;
said Doppler signal is filtered on different Doppler frequency bandwidth so as to assess sensitivity of the Doppler signal on blood velocity;
said region belongs to the brain of said human or animal;
said region belongs to the brain of said human or animal and said series of ultrasound measurements is done through either one of the temporal window, the occipital hole, a trepanation, or artificial thinning down of the skull;
said region belongs to the brain and activation of said region by said stimulus is monitored using a surface electroencephalogram;
said stimulus is a sound;
said region belongs to the retina of at least one first eye of said human or animal;
said region belongs to the retina and activation of said region by said stimulus is monitored using an electroretinogram;
said ultrasound measurements include transmitting and receiving ultrasound waves by said ultrasound probe through the eyelid of said first eye;
said stimulus is luminous;
said luminous stimulus is transmitted through the eyelid of said first eye;
the second eye is open and tracked by video to assess a position of the retina of said first eye, for positioning the ultrasound probe and/or for excluding periods where said retina position of said first eye is improper;
for measuring neurovascular coupling in the nervous system of a human patient, the second eye is open and the patient looks at a visual spot through said second eye during said functional imaging and where the visual spot is either static, or slowly moving to induce a controlled movement of the first eye to perform scanning of the retina;
the patient or animal is anaesthetized during said functional imaging;
at least one response parameter is computed from the hemodynamic response, said at least one response parameter being chosen in the group comprising: a peak value of the hemodynamic response, a risetime computed from the stimulus to the time of the peak value of the hemodynamic response, a fall time computed from the time of the peak value of the hemodynamic response to a minimum value of the hemodynamic response following said peak value;
said at least one response parameter is obtained by fitting a multiparameter function on said hemodynamic response and determining said at least one response parameter on said multiparameter function after fitting;
the method further includes determining whether said hemodynamic response is normal or not;
the method further includes diagnosticating whether said hemodynamic response corresponds to a predetermined disease, in particular a neurodegenerative or cardiovascular disease, for instance Alzheimer disease;
diagnosticating whether the hemodynamic response is normal and/or whether the hemodynamic response corresponds to a predetermined disease, includes comparing said at least one response parameter to a predetermined threshold (i.e., in case of several response parameters: respectively comparing each response parameter to a corresponding predetermined threshold);
the method includes using a neural network trained to determine whether the hemodynamic response is normal and/or to diagnosticate whether the hemodynamic response corresponds to a predetermined disease;
said neural network is trained to determine whether said at least one response parameter corresponds to normal and/or to diagnosticate whether said at least one response parameter corresponds to a predetermined disease;
the method further includes monitoring efficiency of a medical treatment against a predetermined disease, in particular a neurodegenerative or cardiovascular disease, based on said hemodynamic response.

The present disclosure also concerns an apparatus for measuring neurovascular coupling in a nervous system of a human or animal, said nervous system having a vascular network, said apparatus including:
(a) a stimulating device adapted to deliver at least one stimulus to said nervous system, said stimulus activating said nervous system in at least one region thereof, which in turn causes a hemodynamic response in said vascular network in said region;
(b) an ultrasound measuring device adapted to perform a series of at least 10 ultrasound measurements of said region with an ultrasound probe having an array of at least one ultrasound transducer, to obtain hemodynamic Doppler samples of said vascular network in said region, during a recording period of at least 10 seconds including said stimulus, each Doppler sample having a certain Doppler signal;
(c) a computing module adapted to compute, from said series of hemodynamic Doppler measurements, a hemodynamic response to said stimulus in at least one area of interest (20) in said region during said recording period, said hemodynamic response including values of at least one hemodynamic parameter in said vascular network based on said Doppler signals of said Doppler samples of said series.

In embodiments of the system, one may use the following features, alone or in combination:
said computing module is adapted to determine said area of interest automatically based on an activation map of said vascular network, estimated from correlation of said Doppler signals with said stimulus;
said computing module is adapted to determine said area of interest automatically based on Doppler intensity of the Doppler signals;
said computing module is adapted to determine said area of interest automatically based on a B-mode image if said nervous system is a retina;
said ultrasound measurements are Doppler images and said Doppler samples are pixels of said Doppler images;
said ultrasound measurements correspond to one or several lines in a direction of depth from said ultrasound probe;
the ultrasound probe (4) includes a motorization (5) and said ultrasound measuring device (2, 4) is adapted to move said array (6) between measurements to scan at least part of said region;
said series of ultrasound measurements is performed at a rate of at least 1 Doppler image per second;
during said recording period, said series of hemodynamic ultrasound measurements includes at least 50 ultrasound measurements, in particular at least 100 ultrasound measurements;
said recording period is of at least 5 seconds, for instance at least 7 seconds after said stimulus;
said recording period is of at least 5 seconds before and after said stimulus, for instance of at least 7 seconds before and after said stimulus;
said recording period is of at most 20 seconds before and after said stimulus, for instance of at most 10 seconds before and after said stimulus;
said hemodynamic parameter is the Doppler signal;
said hemodynamic parameter is a relative variation of the Doppler signal in said area of interest, relative to a baseline of said Doppler signal;
said computing module is adapted to determine said area of interest based on a preexisting functional map of said region;
said computing module is adapted to determine said area of interest so that it includes at least a pixel of maximum correlation with the stimulus;
said computing module is adapted to determine said area of interest as being constituted by said pixel of maximum correlation and a predetermined number of additional pixels around said pixel of maximum correlation;
said predetermined number of additional pixels is comprised within a radius of 1 to 6 pixels around said pixel of maximum response, for instance within a radius of 2 to 4 pixels;
said computing module is adapted to average said hemodynamic response on said area of interest;
said stimulus has a stimulus duration comprised between 0.05 and 60 seconds, in particular between 0.5 and 1 second, for instance 0.8 seconds;
said stimulus is sensorial, in particular one of: an optical stimulus transmitted through at least one eye, an auditive stimulus transmitted through at least one ear, an odor stimulus transmitted through the nose, a taste stimulus transmitted through the mouth, a contact or shock or electrical stimulus transmitted in particular through the skin;
said apparatus is adapted to repeat said stimulus and said series of ultrasound measurements for n trials and said computing module is adapted to average said hemodynamic response on said n trials, n being an integer larger than 1;
said apparatus is adapted to repeat said stimulus and said series of ultrasound measurements for n trials and said hemodynamic response is used to assess a reproducibility parameter or a quality parameter on said n trials, n being an integer larger than 1;
n is comprised between 10 and 100, for instance comprised between 20 and 60, in particular comprised between 20 and 30;
said array is one of a single transducer, a few transducers (e.g. less than 10), a linear array of transducers (1D matrix), a 2D matrix of transducers and a sparce matrix of transducers;

said ultrasound measurements are based on ultrasensitive Doppler or ultrafast ultrasound imaging with a Pulse Repetition Frequency over 500 Hz;

said ultrasound measurements are based on unfocused ultrasound waves;
said ultrasound measuring device communicates with at least one external device such as video, EEG, ECG, detector of movement of the animal or patient, and said ultrasound measuring device is adapted to control said ultrasound measurements based on signals received from said external device;
said Doppler samples are obtained by one of: standard Doppler and micro-Doppler (see above article of Macé, 2013);
said Doppler signal is based on one of power Doppler, color Doppler, index of vascular resistivity, or any combination thereof;
said computing module is adapted to filter said Doppler signal on different Doppler frequency bandwidth so as to assess sensitivity of the Doppler signal on blood velocity;
said region belongs to the brain of said human or animal;
said region belongs to the brain of said human or animal and said series of ultrasound measurements is done through either one of the temporal window, the occipital hole, a trepanation, or artificial thinning down of the skull;
said region belongs to the brain and activation of said region by said stimulus is monitored using a surface electroencephalogram;
said stimulus is a sound;
said stimulus is luminous;
said region belongs to the retina of at least one first eye of said human or animal and the apparatus further includes a video camera adapted to track the second eye and the apparatus is adapted to assess the position of the retina of said first eye based on said tracking, for positioning the ultrasound probe and/or for excluding periods where said retina position of said first eye is improper;
said region belongs to the retina of at least one first eye of said human and the apparatus further includes a visual spot that may be looked at by the patient through the second eye during said series of ultrasound measurements, the visual spot being either static, or slowly moving to induce a controlled movement of the first eye to perform scanning of the retina;
said computing module is adapted to compute at least one response parameter from the hemodynamic response, said at least one response parameter being chosen in the group comprising: a peak value of the hemodynamic response, a risetime computed from the stimulus to the time of the peak value of the hemodynamic response, a fall time computed from the time of the peak value of the hemodynamic response to a minimum value of the hemodynamic response following said peak value;
said computing module is adapted to obtain said at least one response parameter by fitting a multiparameter function on said hemodynamic response and determining said at least one response parameter on said multiparameter function after fitting;
said computing module is adapted to determine whether said hemodynamic response is normal or not;
said computing module is adapted to diagnosticate whether said hemodynamic response corresponds to a predetermined disease, in particular a neurodegenerative or cardiovascular disease, for instance Alzheimer disease;
said computing module is adapted to diagnosticate whether the hemodynamic response is normal and/or whether the hemodynamic response corresponds to a predetermined disease, includes comparing said at least one response parameter to a predetermined threshold (i.e., in case of several response parameters: respectively comparing each response parameter to a corresponding predetermined threshold);
said computing module includes a neural network trained to determine whether the hemodynamic response is normal and/or to diagnosticate whether the hemodynamic response corresponds to a predetermined disease;
said neural network is trained to determine whether said at least one response parameter corresponds to normal and/or to diagnosticate whether said at least one response parameter corresponds to a predetermined disease;
said computing module is adapted to monitor efficiency of a medical treatment against a predetermined disease, in particular a neurodegenerative or cardiovascular disease, based on said hemodynamic response;
at least one response parameter is computed from the hemodynamic response, said at least one response parameter comprising a peak value of the hemodynamic response, a rise time computed from the stimulus to the time of the peak value of the hemodynamic response, a fall time computed from the time of the peak value of the hemodynamic response to a minimum value of the hemodynamic response following said peak value, said computing module being adapted to compare said at least one response parameter to at least one threshold to diagnosticate said predetermined disease or to monitor said efficiency of said medical treatment;
said computing module is adapted to use a neural network trained to determine whether the hemodynamic response is normal and/or to determine whether the hemodynamic response corresponds to a predetermined disease.

### BRIEF DESCRIPTION OF DRAWINGS

Other features, details and advantages will be shown in the following detailed description and on the figures, on which:

FIG. 1 is a block diagram illustrating an embodiment of an apparatus according to the present disclosure.

FIG. 2 illustrate a possible method of obtaining a series of Doppler images with the apparatus of FIG. 1.

FIG. 3 illustrate part of the apparatus in use, in a specific embodiment where the retina is imaged.

FIG. 4 illustrates the stimulating signal in one specific embodiment.

FIG. 5 shows an example of Doppler image of the retina of a rat obtained with the method of the present disclosure after light stimulation of the eye.

FIG. 6 shows an example of correlation cartography of the retina, enabling to select at least one area of interest.

FIG. 7 shows the doppler signal in the area of interest, with superposed stimulation signal, during n trials.

FIG. 8 shows the average of the Doppler signal on the n trials, with superposed stimulation signal, during n trials.

FIG. 9 shows an example of multiparameter function fitted on the Doppler signal.

FIG. 10 shows the Doppler signals measured on the retina of respectively a sane rat and a transgenic rat modeling Alzheimer disease.

FIG. 11 shows a Doppler image and a correlation cartography of the brain of a rat after light stimulation of the eye.

FIG. 12 shows Doppler signals in the superior colliculus and in the visual cortex, respectively for a sane rat and a transgenic rat modeling Alzheimer disease.

### MORE DETAILED DESCRIPTION

In the Figures, the same references denote identical or similar elements.

The present disclosure proposes a method and apparatus for measuring neurovascular coupling in a nervous system of a human or animal, by functional imaging of the vascular network of said nervous system while delivering a stimulus to said nervous system. The stimulus activates said nervous system in at least one region thereof, which in turn causes a hemodynamic response in said vascular network in said region. The functional imaging enables to obtain a series of hemodynamic Doppler images of the vascular network in said region of the nervous system, showing the hemodynamic response of said region to the stimulus.

The functional imaging may be performed for instance on the retina or on the brain, in which case said region is at least part of the retina or of the brain.

More particularly, the present disclosure concerns functional ultrasound imaging, and notably functional ultrasound ultrafast imaging (see above article of Macé, 2013), which is of particular interest.

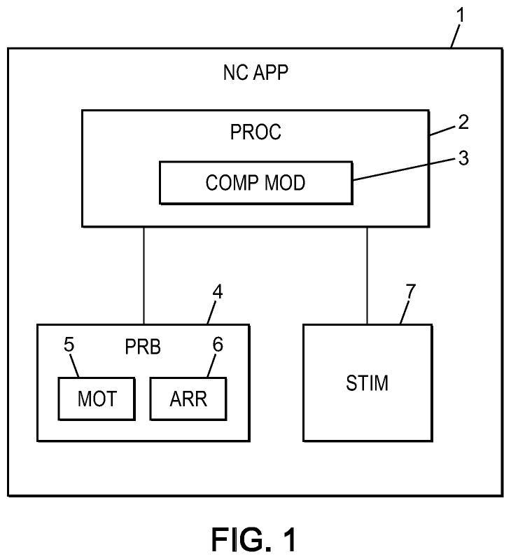

An example of apparatus 1 (NC APP) for measuring neurovascular coupling usable in performing the method according to the present disclosure, is shown on FIG. 1.

The apparatus 1 may include a processor 2 (PROC), for instance a specialized signal processing device controlled a computer or a group of computers, possibly a group of computers including servers.

The processor 2 may include a computing module 3 (COMP), the operation of which will be explained later.

The processor 2 may control a probe 4 (PRB) and a stimulating device 7 (STIM).

The probe 4 may be for instance an ultrasonic probe in the example considered here.

The probe 4 may include an array 6 (ARR) of ultrasonic transducers. The array may be a linear array adapted to generate a 2D image of a slice of the region to be imaged, or a 2D array adapted to generate a 3D image of the region. When the array is a 2D array, it may be a sparce matrix of transducers, as known in the art.

Typical arrays of transducers may include a few hundreds to a few thousand of transducers. The array may also in some examples, be limited to one single transducer adapted to image only one line of the region, in the direction of the depth from the transducer or a few transducers adapted to image respectively lines of the region, in the direction of the depth from the transducer.

The following detailed description is done for the case of a linear or 2D array, so that the apparatus generates Doppler images (more generally: ultrasound measurements) having pixels (more generally: Doppler samples). In the case where the array would include just one transducer or a few transducers, the apparatus would generate an image limited to one line (ultrasound measurement) or a few lines in the direction of depth, the line(s) having pixels (the Doppler samples) and the process would be similar except for the generation of the ultrasound measurements which would not require inclined planar waves of different angles of inclination.

The transducer(s) may be adapted to transmit and receive ultrasound waves having a central frequency comprised for instance between 0.5 and 100 MHz, for instance between 1 and 20 MHz. One example of usable central frequency is 15 MHz.

In certain embodiments, the probe 4 may further include a motorization 5 (MOT) adapted to position the array 6.

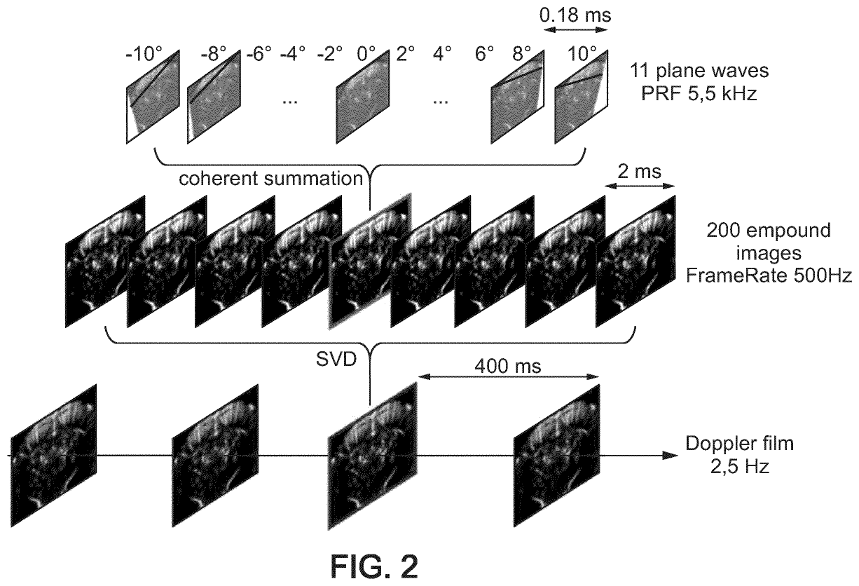

An example of method of functional ultrasound ultrafast imaging, already known in the art and explained for instance in the above article of Macé, 2013, will now be explained with regards to FIG. 2.

The array 6 of transducers may be controlled by processor 2 to transmit planar ultrasonic waves in the region to be imaged and to receive the resulting backscattered ultrasonic waves, at a rate of for instance 5.5 kHz (Pulse Repetition Frequency PRF), i.e. every 18 ms. More generally, the Pulse Repetition Frequency PRF may be over 500 Hz. The received signals are registered as a set of raw data for each transmitted planar ultrasonic wave. The successive transmitted planar waves have propagation direction which are inclined of varying successive angles with regards to the direction of the depth in the region to be imaged, i.e. with regards to the direction normal to the array 6. For each image of the region, a number N of planar ultrasonic waves are successively transmitted with different angles and the N sets of raw data are coherently added to synthesize said image of the region, which is thus a compound image, as explained in said article of Macé 2013. For instance, N may be 11 with angles varying between −10 deg and +10 deg by steps of 2 deg. In the case of N=11 and PRF=5.5 kHz, the rate of the compound images of the region (framerate) is thus 500 Hz. N may be different than 11, in which case the framerate of compound images is different. For instance N=5 may be used.

Based on the successive compound images of the region, hemodynamic Doppler images of the vascular network in said region are then computed by computing module 3. In the example of FIG. 2, 200 successive compound images are used for each hemodynamic Doppler image, so that two successive hemodynamic Doppler images are separated by 400 ms. The rate of hemodynamic Doppler images is thus of 2.5 Hz in the example of FIG. 2. A different number of successive compound images may be used for each hemodynamic Doppler image, in which case the rate of hemodynamic Doppler images is different. For instance, 50 successive compound images could be used for each hemodynamic Doppler image, in which case the rate of hemodynamic Doppler images would be 10 Hz in the example considered here. Generally, the rate of hemodynamic Doppler images is at least 2 Hz.

The hemodynamic Doppler images may be computed for instance by single value decomposition (SVD), as explained by Demene et al. [Demene, C., Robin, J. & Dizeux, A. Transcranial ultrafast ultrasound localization microscopy of brain vasculature in patients. Nat. Biomed. Eng. 5, 219-228 (2021)].

More generally, the hemodynamic Doppler images may be computed by any Doppler technique, including power Doppler, micro-Doppler (as explained in the above article of Mace 2013). The Doppler signal constituting said hemodynamic Doppler images may be for instance power Doppler, color Doppler, index of vascular resistivity, or any combination thereof. Said Doppler signal may be filtered on different Doppler frequency bandwidths so as to assess sensitivity of the Doppler signal on blood velocity.

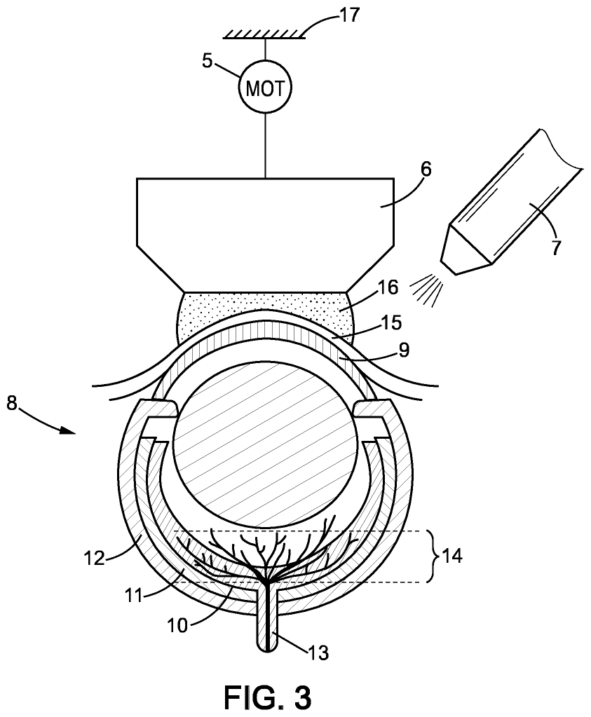

FIG. 3 illustrates how the apparatus may be used for functional ultrasound imaging of the retina 10 of one eye 8. The eye 8 includes inter alia the cornea 9 which may be covered by the eyelid 15. The functional ultrasound imaging may be performed through the closed eyelid 15. The retina 10 belongs to the fundus of the eye, which also includes the choroid 11 and the sclera 12. The optical nerve 13 connects the retina 10 to the brain. The fundus includes a vascular network 14 which is coupled to the retina by neurovascular coupling.

The neurovascular array 6 is coupled to the eye by some gel 16 covering the eyelid 15.

During the functional ultrasonic imaging, it is suitable to cancel or limit movements of the eye.

One way to obtain this result in the case of human imaging is to leave the second eye open and track it by a video camera (not shown) communicating with processor 2, so that processor 2 may assess the position of the retina of the first eye examined by functional ultrasonic imaging. Processor 2 may thus position the array 6 through motorization 5, which connects the array 6 to a support 17, in order to keep the same field of view and/or in order exclude periods where the retina position of said first eye is improper.

Another way to obtain this result in the case of human imaging is to leave the second eye open and have the patient look at a visual spot through said second eye during said functional imaging. The visual spot may be either static, or slowly moving to induce a controlled movement of the first eye, to perform scanning of the retina.

Still another way to obtain this result in the case of human or animal imaging is to have the patient or animal anaesthetized during said functional imaging.

Further, it may be useful to instill the patient or animal eyedrops of a product such as tropicamide prior to the functional ultrasound imaging to induce mydriasis and cycloplegia.

When the array 6 of transducers is linear, the motorization 5 also helps to precisely take successive planar images in neighboring planes. Besides, processor 2 may slightly move the array 6 through motorization 5 back and forth from time to time between ultrasound measurements, to check the positioning of the array 6, and more particularly to check that the planar image includes the region to be imaged or a particular portion of the region to be imaged. More generally, this helps to scan a larger region of the nervous system. In a variant, in case the interrogation ultrasonic beam transmitted by the array is steerable, processor 2 may move said ultrasonic beam between measurements to scan a larger region of the nervous system.

The stimulating device 7 may be of any known type. For instance, the stimulus is sensorial, in particular one of: an optical stimulus transmitted through at least one eye, an auditive stimulus transmitted through at least one ear, an odor stimulus transmitted through the nose, a taste stimulus transmitted through the mouth, a contact or shock or electrical stimulus transmitted in particular through the skin. For instance, stimulating device 7 may be a LED adapted to illuminate the retina of at least one eye of the patient or animal, as illustrated in FIG. 3. The color of the light emitted may be for instance white. The light stimulus may be transmitted to the retina through the closed eyelid of the eye imaged by functional ultrasound imaging.

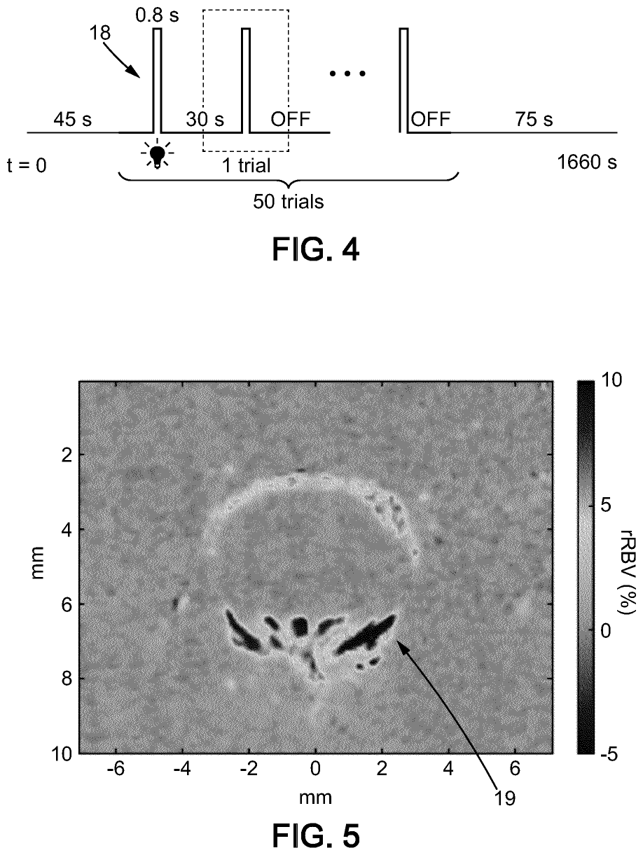

The stimulus may have a stimulus duration comprised between 0.5 and 1 second, for instance 0.8 seconds, as illustrated in FIG. 4 which shows an example of stimulus signal 18 in the case of an optical stimulus. In other embodiments, the stimulus may also be longer, for instance up to 60 seconds.

The computing module 3 may compute and record at least 10 hemodynamic Doppler images of said vascular network in the region, during a recording period of at least 10 seconds including the stimulus.

Said recording period may be of at least 5 seconds, for instance at least 7 seconds after said stimulus.

It may be advantageous to record the hemodynamic Doppler images also before the stimulus, in which case said recording period may be of at least 5 seconds before and after said stimulus, for instance of at least 7 seconds before and after said stimulus.

The recording period may be of at most 20 seconds before and after said stimulus, for instance of at most 10 seconds before and after said stimulus.

In the example of FIG. 4, the recording period is of 30 seconds, including 15 seconds before and after the stimulus. The number of hemodynamic Doppler images computed and registered for one stimulus (i.e. one trial) depends on the recording period and on the rate of hemodynamic Doppler images. For instance, for a recording period T of 30 s and a rate f of hemodynamic Doppler images of 2.5 Hz, the number of hemodynamic Doppler images is T.f=75.

The stimulus may be repeated regularly while the functional imaging is computing hemodynamic Doppler images. The number n of trials may be for instance of 50 in the example of FIG. 4 but may be different. More generally, n may be comprised between 10 and 100, for instance comprised between 20 and 60, in particular comprised between 20 and 30. For instance, the number of trials may be reduced to 25.

The series of trials may be preceded by an initial period without stimulus of for instance 45 s and followed by a final period without stimulus of for instance 75 s, in the example of FIG. 4.

FIG. 5 illustrates an example of hemodynamic Doppler image of the retina of a rat, computed by computing module 3 after activation by a light stimulus. The Doppler image shows a hemodynamic parameter based on the Doppler signal.

The signal of each pixel in the Doppler image may be the Doppler signal itself or, more generally, a signal based on said Doppler signal.

In the case of FIG. 5, the hemodynamic parameter shown on the Doppler image is the relative Retinal Blood Volume rRBV. The Doppler signal coming directly from the functional ultrasound imaging corresponds to the retinal blood volume, i.e. the blood volume having an axial velocity higher than a predetermined threshold (for instance 4 mm/s). The relative blood volume corresponds to the relative variation of Retinal Blood Volume RBV compared to the baseline BL, in % (i.e., rRBV=(RBV−BL)/BL, wherein BL is the blood volume in the absence of stimulus.

FIG. 5 enables to clearly see the areas 19 where the neurovascular coupling is high in the vascular network 14 of the retina, after activation by a light stimulus.

Computing module 3 is adapted to select an area of interest, where the neurovascular coupling is maximum.

The area of interest may be automatically determined by computing module 3 based on an activation map of said vascular network, estimated from correlation of said Doppler signals with said stimulus (i.e. said area of interest is determined as a set of pixel where the Doppler signal is sufficiently correlated with the stimulus).

For instance, the area of interest may include at least a pixel of maximum correlation with the stimulus. Particularly, said area of interest may be constituted by said pixel of maximum correlation and a predetermined number of additional pixels around said pixel of maximum correlation.

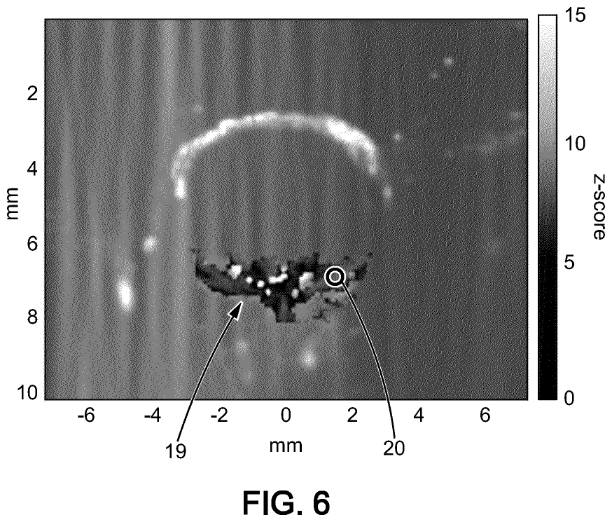

Said predetermined number of additional pixels may be comprised within a radius of 1 to 6 pixels around said pixel of maximum response, for instance within a radius of 2 to 4 pixels. FIG. 6 illustrates such area of interest 20, determined from a mapping of correlation. The correlation parameter used on FIG. 6 is called z-score, computed according to the known Global Linear Model (GLM), widely used in particular in the field of functional MRI.

In other variants, said predetermined number of additional pixels may be for instance a square of pixels, e.g. 7*7 pixels.

The determination of the area of interest based on correlation may be carried out after averaging the correlation maps on the n trials, or, usually, by using the dataset from the n trials.

In other embodiments, the computing module 3 is adapted to determine said area of interest automatically based on Doppler intensity of the Doppler signals, thus targeting the area of maximum blood flow.

In other embodiments, the computing module 3 is adapted to determine said area of interest automatically based on a B-mode image, which may be sufficient if said nervous system is a retina.

In other embodiments, the computing module 3 is adapted to determine said area of interest automatically based on an external neuronavigation device.

In still other variants, the selection of the area of interest may also take into account a preexisting functional cartography of the region to be imaged, indicating which areas of the nervous system are activated by a given stimulus. This approach may be more useful for the functional imaging of the brain, using for instance known brain atlases. Such maps are available for the human brain and some animal brains, e.g. the rat.

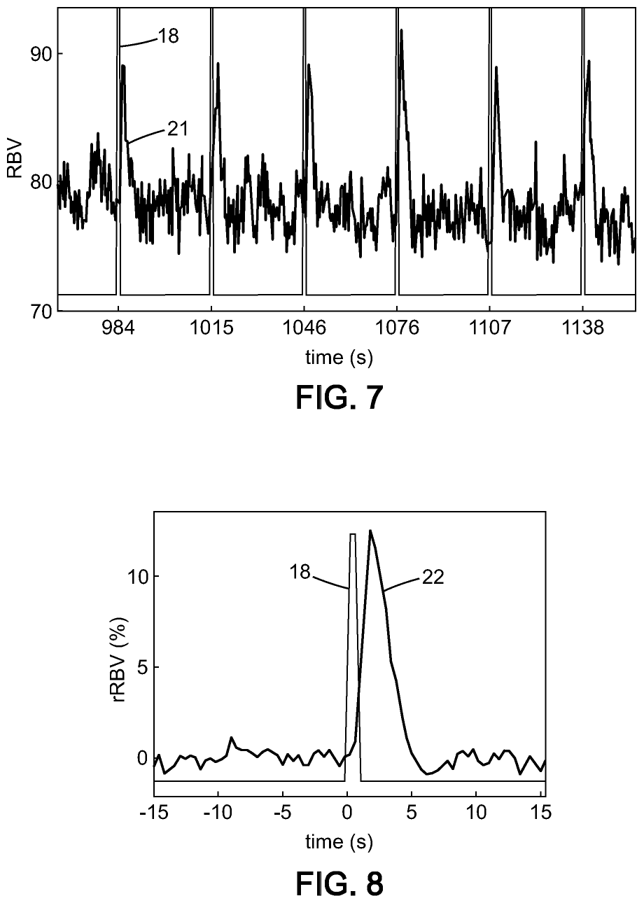

Once the area of interest is determined, the computing module may average the hemodynamic signal on the area of interest, thus obtaining the curve 21 of hemodynamic response shown on FIG. 7, through the n trials. The hemodynamic parameter shown on FIG. 7 is the Retinal Blood Volume RBV but could be rRBV as previously described or another parameter.

The hemodynamic response may then be averaged on the n trials, to obtain an average curve 22 as shown on FIG. 8 in the case where the hemodynamic parameter is rRBV.

The computation of the hemodynamic response 22 over the n trials may also be used to assess the reproducibility or quality of the hemodynamic parameters estimation over said n trials, for instance by computation of statistical parameters such as variance and standard deviation.

The shape of the hemodynamic response, especially after average on the n trials (curve 22 of FIG. 8), may be used as a reliable biomarker of certain health disorders. Thus, based on the hemodynamic response, it is possible to reliably determine whether the neurovascular coupling is normal or not, and to use the neurovascular coupling as a biomarker of diseases such as certain neurodegenerative or cardiovascular diseases, for instance Alzheimer disease.

To this end, computing module 3 may be is adapted to compute at least one response parameter from the hemodynamic response, said at least one parameter being chosen in the group comprising:
a maximum value of the hemodynamic response,
a risetime computed from the stimulus to the time of the maximum value of the hemodynamic response,
a fall time computed from the time of the maximum value of the hemodynamic response to a minimum value of the hemodynamic response following said maximum value.

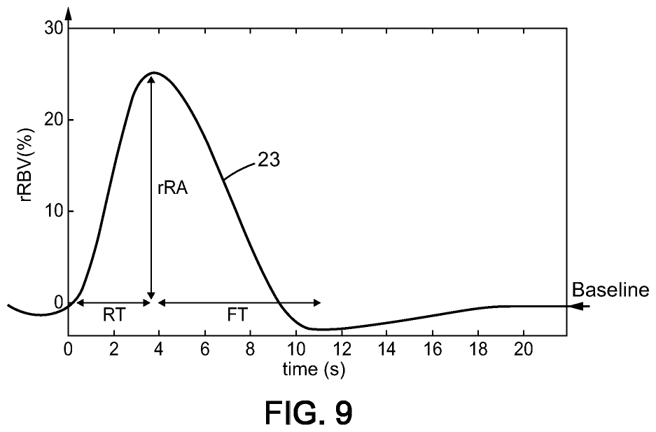

These parameters may be computed by said computing module by fitting a multiparameter function on said hemodynamic response and determining said at least one parameter on said multiparameter function after fitting. For instance, as shown on FIG. 9 in the n, the multiparameter function may be constituted by 4 half-period of cosine functions. FIG. 9 indicates how are determined, on the fitted multi-parameter function, the maximum value rRA of the hemodynamic response, the rise time RT and the fall time FT.

For determining whether the hemodynamic response is normal and/or whether the hemodynamic response corresponds to a predetermined disease, computing module 3 may compare said parameters to predetermined thresholds.

In a variant, for determining whether the hemodynamic response is normal and/or whether the hemodynamic response corresponds to a predetermined disease, computing module 3 may use a neural network trained to determine whether the hemodynamic response is normal and/or to determine whether the hemodynamic response corresponds to a predetermined disease. This determination by the neural network may be carried out directly on the average curve 22 of the hemodynamic response, or on the fitted multi-parameter function, or on the parameters as discussed above.

What has been explained above for measuring the neurovascular coupling in the retina is applicable also for measurement in the brain, except for the features of the method and apparatus which are specific to functional imaging of the eye. In the case of functional ultrasound imaging of the brain, it is useful to transmit and receive the ultrasonic waves through either one of the temporal window, the occipital hole, a trepanation, or an artificial thinning down of the skull. In the case of functional ultrasound imaging of the brain, the stimulus may be advantageously sound.

The apparatus as described above may also be used to monitor efficiency of a medical treatment against a predetermined disease, in particular a neurodegenerative or cardiovascular disease, based on the hemodynamic response. To this end, the hemodynamic response may be measured at different points of time at least before and after the medical treatment, possibly including during the medical treatment, to determine whether the medical treatment improves neurovascular coupling. This monitoring can also be done by comparing said at least one response parameter to at least one threshold, or by using a neural network, as described above.

In all embodiments, the functional imaging may be controlled by signals from external devices such as video, EEG, ECG, detector of movement of the animal or patient, etc.

Actual activation and the level of activation of the imaged region of the nervous system may be monitored for instance by surface electroencephalogram in the case of the brain and by electroretinogram in the case of the retina.

Specific examples will now be presented on the particular case of detection of Alzheimer disease by measuring the neurovascular coupling in the retina and brain of the rat, comparing normal rats and genetically modified TgF344-AD rats which constitute a good murine model simulating Alzheimer disease.

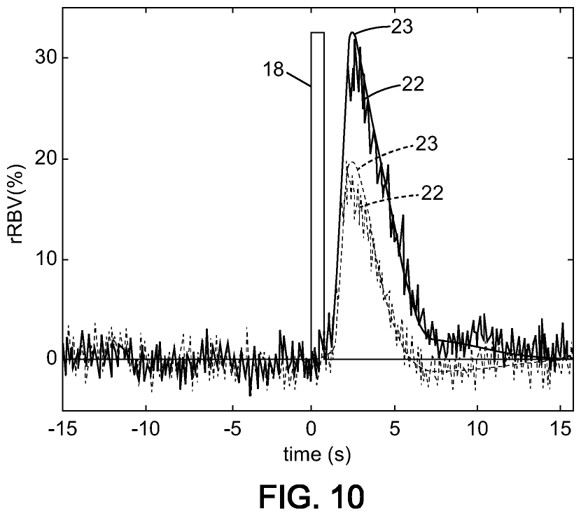

The hemodynamic response in the retina was determined for 6 normal rats and 6 TgF344-AD rats, as explained above. FIG. 10 shows the average hemodynamic response 22 on 50 trials and the fitted multiparameter function 23 averaged on the 6 normal rats (solid lines) and averaged on the 6 TgF344-AD rats (dotted lines). FIG. 10 shows that the maximum value rRA is substantially increased the TgF344-AD rats, thus confirming the possibility to use rRA as a biomarker of Alzheimer disease in this example.

A similar study was carried out for the hemodynamic response in the brain was determined for 2 normal rats and 3 TgF344-AD rats. The functional ultrasound imaging was carried out through a thinned portion of the skull. More specially, the hemodynamic response was computed in the superior colliculus and in the virtual cortex.

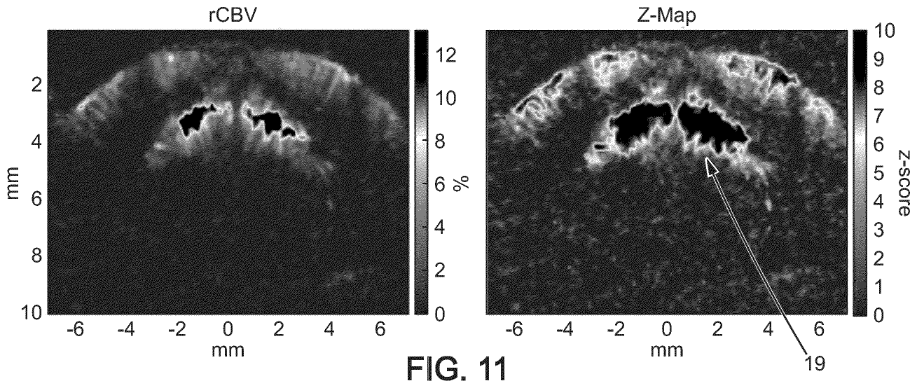

FIG. 11 shows images of respectively the hemodynamic response (relative Cerebral Blood Volume—rCBV) and the correlation parameter (z-score) in the superior colliculus and in the virtual cortex.

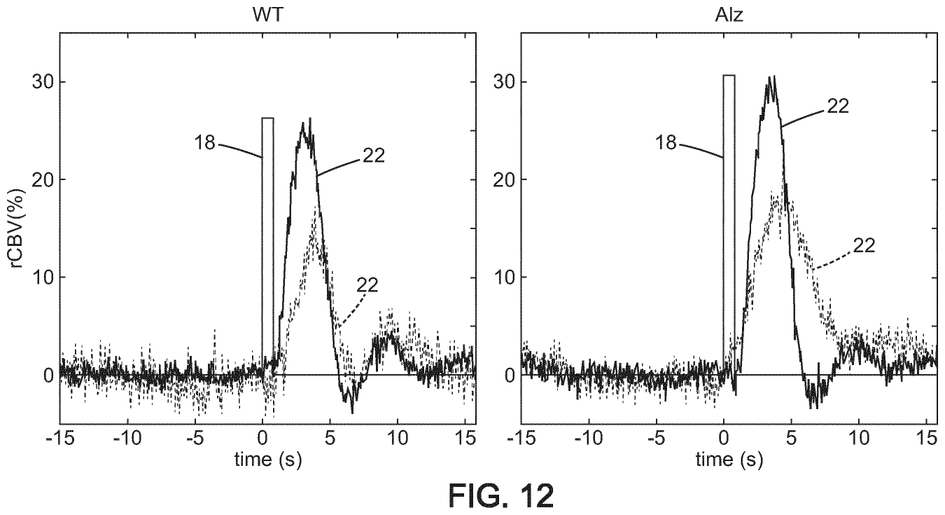

FIG. 12 shows the average hemodynamic response 22 on 50 trials in the superior colliculus (solid lines) and in the visual cortex (dotted lines), averaged respectively for normal rats (WT) and averaged on the TgF344-AD rats (Alz). FIG. 12 shows that the maximum value of rCBV is substantially increased for the TgF344-AD rats, thus again confirming the possibility to use the maximum value rRA as a biomarker of Alzheimer disease in this example.

1. Method for measuring neurovascular coupling in a nervous system of a human or animal, said nervous system having a vascular network, said method including:
(a) delivering at least one stimulus to said nervous system, said at least one stimulus activating said nervous system in at least one region thereof, which in turn causes a hemodynamic response in said vascular network in said at least one region;
(b) performing a series of at least 10 ultrasound measurements of said at least one region with an ultrasound probe having an array of at least one ultrasound transducer, to obtain hemodynamic Doppler samples of said vascular network in said at least one region, during a recording period of at least 10 seconds including said stimulus, each Doppler sample having a certain Doppler signal;
(c) computing, from said series of hemodynamic Doppler measurements, a hemodynamic response to said at least one stimulus in at least one area of interest in said at least one region during said recording period, said hemodynamic response including values of at least one hemodynamic parameter in said vascular network based on said Doppler signals of said Doppler samples of said series.

2. Method according to claim 1, wherein said at least one area of interest is determined automatically based on:
an activation map of said vascular network, estimated from correlation of said Doppler signals with said at least one stimulus;
or Doppler intensity of the Doppler signals;
or a B-mode image if said nervous system is a retina;
or an external neuronavigation device.

3. Method according to claim 1, wherein either said ultrasound measurements are Doppler images and said Doppler samples are pixels of said Doppler images, or said ultrasound measurements correspond to one or several lines in a direction of depth from said ultrasound probe.

4. Method according to claim 1, wherein an interrogation ultrasonic beam transmitted by the ultrasound probe is moved between measurements to scan at least part of said at least one region.

5. Method according to claim 1, wherein said series of ultrasound measurements is performed at a rate of at least 1 Doppler image per second.

6. Method according to claim 1, wherein said hemodynamic response is averaged on said at least one area of interest.

7. Method according to claim 1, wherein said steps (a) and (b) are repeated for n trials and said hemodynamic response is averaged on said n trials, n being an integer comprised between 10 and 100.

8. Method according to claim 1, wherein said steps (a) and (b) are repeated for n trials and said hemodynamic response is used to assess a reproducibility parameter or a quality parameter on said n trials, n being an integer comprised between 10 and 100.

9. Method according to claim 8, wherein said at least one region belongs either to the brain of said human or animal, or to the retina of a first eye of said human or animal.

10. Method according to claim 9, wherein:
either said at least one region belongs to the brain and activation of said at least one region by said at least one stimulus is monitored using a surface electroencephalogram;
or said at least one region belongs to the retina and activation of said at least one region by said at least one stimulus is monitored using an electroretinogram.

11. Method according to claim 9, wherein said at least one region belongs to the retina and said ultrasound measurements include transmitting and receiving ultrasound waves by said ultrasound probe through an eyelid of said first eye.

12. Method according to claim 11, wherein said at least one stimulus is luminous and said luminous stimulus is transmitted through the eyelid of said first eye.

13. Method according to claim 11, wherein:
either a second eye is open and tracked by video to assess a position of the retina of said first eye, for positioning the ultrasound probe and/or for excluding periods where said retina position of said first eye is improper;
or, for measuring neurovascular coupling in the nervous system of a human patient, the second eye is open and the patient looks at a visual spot through said second eye during said functional imaging and where the visual spot is either static, or slowly moving to induce a controlled movement of the first eye to perform scanning of the retina.

14. Method according to claim 1, wherein at least one response parameter is computed from the hemodynamic response, said at least one response parameter being chosen in the group comprising: a peak value of the hemodynamic response, a rise time computed from the stimulus to the time of the peak value of the hemodynamic response, and a fall time computed from the time of the peak value of the hemodynamic response to a minimum value of the hemodynamic response following said peak value.

15. Method according to claim 14, wherein said at least one response parameter is obtained by fitting a multiparameter function on said hemodynamic response and determining said at least one response parameter on said multiparameter function after fitting.

16. Apparatus for measuring neurovascular coupling in a nervous system of a human or animal, said nervous system having a vascular network, said apparatus including:
(a) a stimulating device adapted to deliver at least one stimulus to said nervous system, said at least one stimulus activating said nervous system in at least one region thereof, which in turn causes a hemodynamic response in said vascular network in said at least one region;
(b) an ultrasound measuring device adapted to perform a series of at least 10 ultrasound measurements of said at least one region with an ultrasound probe having an array of at least one ultrasound transducer, to obtain hemodynamic Doppler samples of said vascular network in said at least one region, during a recording period of at least 10 seconds including said stimulus, each Doppler sample having a certain Doppler signal;
(c) a computing module adapted to compute, from said series of hemodynamic Doppler measurements, a hemodynamic response to said stimulus in at least one area of interest in said at least one region during said recording period, said hemodynamic response including values of at least one hemodynamic parameter in said vascular network based on said Doppler signals of said Doppler samples of said series.

17. Apparatus according to claim 16, wherein said computing module is adapted to diagnose whether said hemodynamic response corresponds to a predetermined disease.

18. Apparatus according to claim 16, wherein said computing module is adapted to monitor efficiency of a medical treatment against a predetermined neurodegenerative or cardiovascular disease, based on said hemodynamic response.

19. Apparatus according to claim 17, wherein at least one response parameter is computed from the hemodynamic response, said at least one response parameter comprising a peak value of the hemodynamic response, a rise time computed from the stimulus to the time of the peak value of the hemodynamic response, a fall time computed from the time of the peak value of the hemodynamic response to a minimum value of the hemodynamic response following said peak value, said computing module being adapted to compare said at least one response parameter to at least one threshold to diagnose said predetermined disease or to monitor said efficiency of said medical treatment.

20. Apparatus according to claim 17, wherein said computing module is adapted to use a neural network trained to determine whether the hemodynamic response is normal and/or to determine whether the hemodynamic response corresponds to the predetermined disease.
